(19) United States
(12) Patent Application Publication
(10) Pub. No.: US 2026/0325332 A1
(43) Pub. Date: Oct. 22, 2026

(54) Stent Delivery System

(71) Applicant: Micro Medical Solutions, Inc., Woburn, MA (US)

(72) Inventors: Gregory Sullivan, Woburn, MA (US); Nikolaos D. Pappas, Woburn, MA (US); Brendan P. LaBrecque, Woburn, MA (US)

(21) Appl. No.: 19/365,397

(22) Filed: Oct. 22, 2025

Related U.S. Application Data
(63) Continuation-in-part of application No. 18/970,095, filed on Dec. 5, 2024.
(60) Provisional application No. 63/606,400, filed on Dec. 5, 2023.
(60) Provisional application No. 63/710,168, filed on Oct. 22, 2024.

(51) Int. Cl. A61F 2/966; A61M 25/00; A61M 25/01; A61M 25/06; A61M 39/00; A61M 39/06

(52) U.S. Cl. CPC A61F 2/966; A61M 25/0045; A61M 25/005; A61M 25/0097; A61M 25/0108; A61M 25/0662; A61M 39/06; A61M 2025/0047; A61M 2039/0009; A61M 2039/0626; A61M 2205/32; A61M 2210/12

(57) ABSTRACT

Disclosed is a stent delivery system, including a delivery sheath catheter, a stabilizer, and a tuohy borst sideport. The delivery sheath catheter includes an inner lubricious liner, a reinforcement layer, and a wear-resistant outer jacket with an atraumatic distal tip, and a distal radiopaque marker band to facilitate visualization under fluoroscopy. The stabilizer, internally concentric within the delivery sheath catheter, includes an inner lumen for a 0.014-inch guidewire, a flat face for stent deployment, a proximal radiopaque split marker band to facilitate visualization under fluoroscopy, and a reinforced shaft. The preloaded stent is positioned between the delivery sheath catheter inner liner and the stabilizer lumen at the distal end of the delivery system. The stent delivery system, with a crossing profile of 3.20 to 3.89 French and a length between 40 to 220 cm, is configured for navigation through tight diseased vasculature to deploy the stent at distal lesion sites.

### CROSS REFERENCE TO RELATED APPLICATION

This patent application is a continuation-in-part application which claims the benefit of and takes priority from U.S. Utility patent application Ser. No. 18/970,095 filed on Dec. 5, 2024, which in turn claims the benefit of and takes priority from U.S. Provisional Patent Application Ser. Nos. 63/710,168 filed on Oct. 22, 2024 and 63/606,400, filed on Dec. 5, 2023, the contents of which are herein incorporated by reference.

### BACKGROUND OF THE INVENTION

### Field of the Invention

The invention relates to medical devices, specifically to a minimally invasive stent delivery system designed for percutaneous access, trackability, and deployment into diseased vascular anatomies. More particularly, the invention pertains to a low crossing profile stent delivery system configured to navigate tight and tortuous arterial or venous pathways to deliver a self-expanding stent to distal lesion sites. The stent delivery system incorporates features to enhance flexibility, trackability, and precise placement in challenging anatomical conditions.

### Description of the Related Art

### Need for Medical Intervention:

Within the body, a stenosis or abnormal narrowing may occur in a blood vessel or other tubular organ or structure. In a blood vessel, a stenosis or dissection from prior device use such as a balloon angioplasty, may obstruct blood. Within the art, stent devices may be used to treat stenoses. This invention is applicable to the delivery of such devices.

### Vascular Stents:

A vascular stent is a small, mesh-like tube that is placed into a stenosed blood vessel to keep it open and restore blood flow. The device acts as a scaffold, supporting the vessel walls and preventing them from collapsing or otherwise causing obstruction. There exist a wide variety of vascular stents used for various applications. Vascular stents are typically deployed under fluoroscopy using stent delivery system catheters. Such a stent delivery system often includes a delivery sheath catheter and an interior stabilizer (pusher). Once placed, stents may be either self-expanding or balloon expandable. Self-expanding stents are often made from super elastic nitinol tubes or wire and utilize the inherent spring-like properties that can undergo large strains without permanent deformation when displacing from a loaded compressed state while in the stent delivery system and self-expanding during deployment inside a diseased vessel. In contrast, balloon-expandable stents require inflation of an integrated balloon to achieve their deployed configuration.

The design of the delivery sheath catheter is critical to the performance of a stent deployment catheter. A delivery sheath catheter is subject to a number of often conflicting design requirements such as flexibility, kink resistance, axial tensile and compressive strength, minimized exterior diameter, and maximized interior diameter. In particular, there is a balance between a need for flexibility and a need for strength. Therefore, a need remains for improved medical devices such as stent deployment catheters that are optimized for performance.

### Unmet Need(s) in the Prior Art:

Existing stent delivery systems face limitations in navigating extremely tight, tortuous, or distal vascular anatomies. These include inadequate balance between low profile and structural integrity, suboptimal friction management for stent loading and deployment. There remains a need for a stent delivery system that provides reliable low-profile tracking over standard 0.014 guidewires. Improved designs are sought to optimize these aspects, enabling safer, more efficient deployment of self-expanding stents in challenging anatomies.

In endovascular interventions, the Pin and Pull technique is sometimes used for deploying self-expanding stents. The stent is held in a compressed loaded state against the internal surface of an outer sheath and constrained in the interior by an inner catheter shaft. Retraction of the delivery sheath catheter via a pull-grip at the proximal end of the delivery sheath catheter while simultaneously keeping the inner shaft pinned” will result in stent deployment. Ancillary accessories are necessary to enable stent delivery system use, including guidewires ranging in diameter of 0.014″, 0.018″, or 0.035″, as well as 4, 5, 6 or 7 French guiding sheath, and introducer sheaths.

### SUMMARY OF THE INVENTION

The invention, as illustrated herein, is clearly not anticipated, rendered obvious, or even present in any of the prior art mechanisms, either alone or in any combination thereof. A novel design is disclosed for a stent delivery system used with a preloaded self-expanding stent.

The invention pertains to a low crossing profile stent delivery system configured to navigate tight and tortuous arterial or venous pathways to deliver a self-expanding stent to distal lesion sites. The stent delivery system incorporates features to enhance flexibility, trackability, and precise placement in challenging anatomical conditions. The stent delivery system, with a crossing profile of 3.20 French to 3.89 French and a length between 40 to 220 cm, is configured for navigation through tight and tortuous diseased vasculature to deploy the stent at distal lesion sites reliably.

The stent delivery system described may be used to, but not limited to, deployment of the MicroStent, a self-expanding woven nitinol stent which is intended to maintain patency in vessels of 2.0-4.5 mm diameter, for lesions located below the knee having a length of 8 mm to 119 mm. The MicroStent is indicated for the treatment of critical limb threatening ischemia (CLTI) in the lower leg using the femoral or pedal artery as the interventional percutaneous access point.

The stent delivery system includes an outer delivery sheath catheter and interior stabilizer (pusher) catheter to hold a self-expanding stent in a loaded state. The delivery sheath catheter comprises a proximal hub, elongated shaft, and distal marker band. The proximal hub is used to flush and de-air the delivery sheath catheter prior to use. The elongated shaft allows the delivery sheath catheter to track over a guidewire to deliver the stent to the target lesion. The distal radiopaque marker band provides a visual reference under fluoroscopy for accurate placement of the stent.

The delivery sheath catheter offers the advantages of low profile, high tensile strength (for example in excess of, 20N), and good trackability for placement of vascular stents in small, tortuous blood vessels.

It is a further object of the present invention to provide a delivery sheath catheter with a low friction interior surface to allow for easier deployment of stents during medical procedures. It is a further object of the present invention to provide a delivery sheath catheter and stabilizer configured to retain the stent in a loaded state, ready to use.

The stent delivery system is configured to maintain structural integrity and responsiveness during introductory forces, to maintain structural integrity and responsiveness while advancing percutaneously through a vascular anatomy, to maintain structural integrity and responsiveness during deployment of a preloaded stent at a target, and to maintain structural integrity and responsiveness during stent delivery system withdrawal from a patient.

A primary advantage to this stent delivery system is on low profile, while still being high-robustness and low elongation. High robustness is defined here as having a low chance of kinking when subjected to a 0.75″ bend diameter, and if kinked not being damaged to the point being unusable, and abrasion resistant, and able to exceed 20N force without damage when loaded axially. Low elongation is described as both not exceeding 10% elongation at 10N of axial load, and being recoverable to original not loaded state (not sustaining deformation).

It is a further object of the present invention to provide a stabilizer catheter with an interior main lumen that is sufficiently robust, stiff and universally compatible with any 0.014″ guidewire, of adequate length (for example, but not limited to 180 cm length, 300 cm length, etc.) having stock lubricous coatings (for example, water activated hydrophilic coatings, or dry lubricious PTFE coatings) for dependable stent delivery system tracking to a target distal lesion during medical procedures.

It is a further object of the present invention to provide a delivery sheath catheter and a stabilizer shaft that work together as system to maintain superior flexibility, kink resistance, and axial tensile and compressive strength when traversing tortuous paths during medical procedures.

It is a further object of the present invention to provide a delivery sheath catheter with a distal radiopaque distal marker band providing a visual reference of the distal position of the stent under fluoroscopy image guidance.

It is a further object of the present invention to provide a stabilizer catheter with a proximal radiopaque split marker band providing a visual reference of the proximal position of the stent under fluoroscopy image guidance.

It is a further object of the present invention to provide a delivery sheath catheter with a uniform stiffness, or a customizable stiffness profile along its length to allow for a multitude of clinical applications.

It is a further object of the present invention to provide a delivery sheath catheter with a soft distal region for increased maneuverability during medical procedures.

It is a further object of the present invention to provide a delivery system with a low overall crossing profile, to allow for easier maneuverability and increased usages during medical procedures.

There has thus been outlined, rather broadly, the more important features of a stent delivery system, the description thereof that follows may be better understood, and in order that the present contribution to the art may be better appreciated. There are additional features of the system that will be described hereinafter and which will form the subject matter of the claims appended hereto.

In this respect, before explaining at least one embodiment of the system in detail, it is to be understood that the system is not limited in its application to the details of construction, assembly and arrangements of the components set forth in the following description or illustrated in the drawings. The system is capable of other embodiments and of being practiced and carried out in various ways. Also, it is to be understood that the phraseology and terminology employed herein are for the purpose of description and should not be regarded as limiting.

These together with other objects of the system, along with the various features of novelty, which characterize the system, are pointed out with particularity in the claims annexed to and forming a part of this disclosure. For a better understanding of the system, its operating advantages and the specific objects attained by its uses, reference should be made to the accompanying drawings and descriptive matter in which there are illustrated preferred embodiments of the system.

The foregoing has outlined the more pertinent and important features of the present system in order that the detailed description of the system that follows may be better understood, and the present contributions to the art may be more fully appreciated. It is of course not possible to describe every conceivable combination of components and/or methodologies, but one of ordinary skill in the art may recognize that many further combinations or permutations are possible. Accordingly, the novel architecture described below is intended to embrace all such alterations, modifications, and variations that fall within the spirit and scope of the appended claims.

### BRIEF DESCRIPTION OF THE DRAWINGS

Advantages of the present system will be apparent from the following brief description of exemplary embodiments thereof; which description should be considered in conjunction with the accompanying drawings. Having thus described the invention in general terms, reference will now be made to the accompanying drawings, which are not necessarily drawn to scale. The system may be more completely understood in consideration of the following detailed description of various embodiments of the system in connection with the accompanying drawings, in which:

FIG. 1 is a longitudinal side view of the stent delivery system with a preloaded self-expanding stent loaded at the distal end.

FIG. 2 is a side cross sectional view of the stent delivery system distal end, showing the distal portion of the self-expanding stent present in the space allotted by the stabilizer and the delivery sheath.

FIG. 3 is a side cross sectional view of the stent delivery system distal end, showing the proximal portion of the self-expanding stent present in the space allotted by the stabilizer and the delivery sheath catheter and abutting the stabilizer backstop feature.

FIG. 4 is a side cross sectional view of the stent delivery system proximal end, showing the proximal end of the delivery sheath catheter 200 mounted permanently to the tuohy borst sideport 300, and clamping to the exterior portion of the stabilizer 400.

FIG. 5 is a side cross sectional view of the stent delivery system proximal end, showing the stabilizer 400 proximal hub.

FIG. 6A is a perspective side view of stent delivery system with a stent loaded in the distal end of the stent delivery system.

FIG. 6B is a perspective side view of stent delivery system with a partially deployed from the distal end of the stent delivery system.

FIG. 6C is a perspective side view of stent delivery system with a stent deployed from the end.

FIG. 7A is a perspective side view of stent delivery system proximal end with a stent presumed to be loaded at the distal end.

FIG. 7B is a perspective side view of stent delivery system proximal end with a stent presumed to be deployed from the distal end.

FIG. 8 is a perspective view of the various stent delivery system length embodiments.

FIG. 9A is a perspective view of delivery system sheath construction embodiment made with a solid super elastic Nitinol reinforcement layer.

FIG. 9B is a perspective view of delivery system sheath construction embodiment made a metallic hypotube having a spiral laser interruption cut reinforcement layer.

FIG. 9C is a perspective view of delivery system sheath construction embodiment made with a braided reinforcement layer.

FIG. 10 is a perspective view of the various stent length and diameter embodiments.

FIG. 11 is a perspective view of a stent delivery system fitted with an embodied braided delivery sheath catheter construction, showing robustness of the system.

FIG. 12A is a perspective view of a stent delivery system having a small crossing profile, allowing passage through an off-the-shelf 4 Fr Introducer Sheath.

FIG. 12B is a perspective view of a stent delivery system having a small crossing profile and deploying a preloaded stent.

FIG. 13A is a perspective view of an inner lumen preferred embodiment.

FIG. 13B is an exploded perspective view of the inner lumen preferred embodiment.

FIG. 13C is a perspective view of an inner lumen alternate embodiment.

FIG. 13D is an exploded perspective view of the inner lumen alternate embodiment.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE SEVERAL EMBODIMENTS

For the following defined terms, these definitions shall be applied, unless a different definition is given in the claims or elsewhere in this specification. All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein reference numerals indicate like elements throughout the several views. The drawings, which are not necessarily to scale, depict illustrative embodiments of the claimed invention.

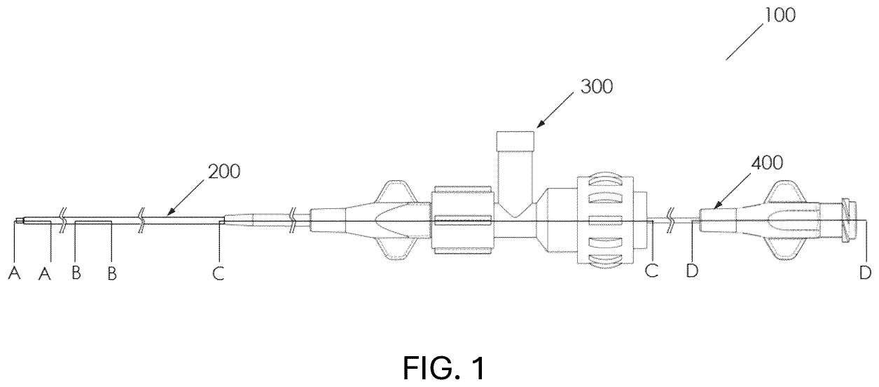

FIG. 1 illustrates a side view of one embodiment of the stent delivery system 100 with a preloaded self-expanding 500 loaded at the distal end (not shown). The stent delivery system further comprises a delivery sheath catheter 200, tuohy borst sideport 300, and a stabilizer 400. “Proximal” end refers to the end closer to an entry location outside the body, which is shown from the right side, whereas “distal” end refers to the farthest end from the entry location, and shown from the left side.

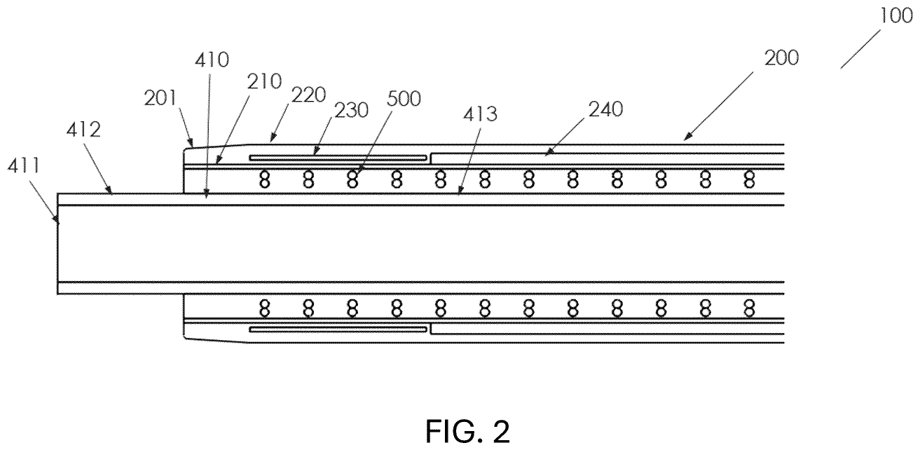

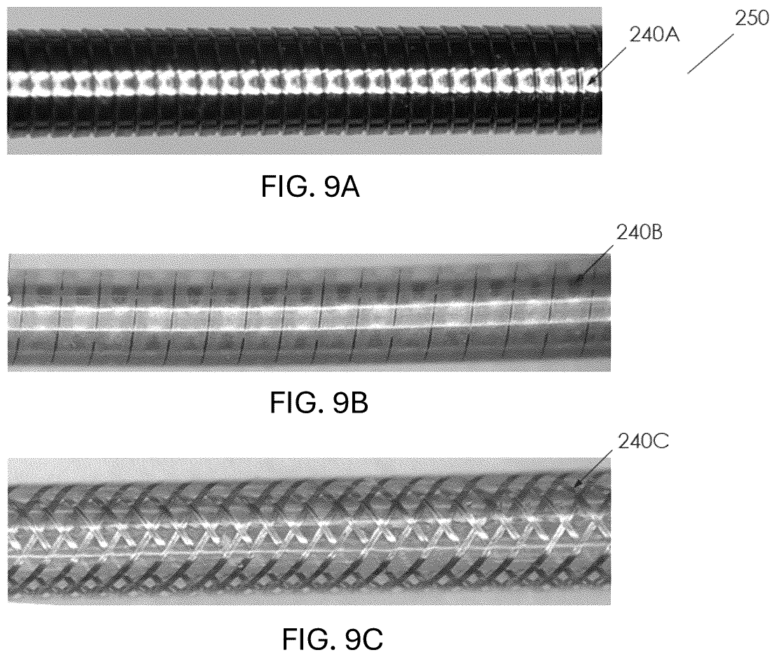

FIG. 2 illustrates a side cross sectional view along line A-A of the stent delivery system 100 shown in FIG. 1, showing the distal portion of the stent 500 in the loaded state within the space allotted by the stabilizer 400 and the delivery sheath catheter 200. In one embodiment, the stabilizer 400 herein comprises an inner lumen 410, which may be comprised of a distal cut polymer tube with a round open internal diameter of 0.0175″+/−0.0005″ spanning the entire length of the stent delivery system interrupted. In one embodiment, the inner lumen 410 may have a wall thickness of 0.0025″ and have a break strength of 20N or greater, and an elongation of less than 10% when axially loaded at 10N. In one embodiment, the inner lumen 410 may have an internal diameter that is concentric with the outside diameter by 90% or greater. In one embodiment, in order to ensure low friction passage of a 0.014″ guidewire 700 (not shown), the inner lumen 410 is prepared at the distal tip having a clean square cut end 411. In one embodiment, the inner lumen 410 has a protruded length 412 of 1 mm to 3 mm from the atraumatic tip 201, to allow a clinician to locate and feed the stent delivery system 100 onto a 0.014″ guidewire 700 (not shown). In one embodiment, the inner lumen 410 has an outer surface 413 that is abrasion resistant and supportive of the stent 500 in the loaded state. The inner lumen 410 extends the full length of the stent delivery system as a single, continuous, uninterrupted piece. The delivery sheath catheter 200 may comprise an atraumatic tip 201, where only a lubricious inner liner 210 and soft wear resistant jacket polymer 220 laminated together are present. An atraumatic tip reduces forces on interior vessel walls during stent delivery system advancement to increase trackability. The stent 500 is loaded with inherent spring tension up against the lubricious inner liner 210, and the edge is within the length of a radiopaque marker band 230. In one embodiment the radiopaque marker band 230 is made of 90% platinum and 10% iridium and has an outside diameter the is coincident with the outside diameter of a reinforcement layer 240. The delivery sheath catheter 200, over the majority of the device length, comprises three layers: the lubricious inner liner 210, the reinforcement layer 240, and the soft wear resistant jacket polymer 220. The lubricious inner liner 210 may be comprised of any suitable polymeric material. In various embodiments, the lubricious inner liner 210 may comprise a lubricious polymer such as polytetrafluoroethylene (PTFE) or fluorinated ethylene propylene (FEP), which may be etched to enhance thermal bonding to the shaft reinforcement layer 240. In one embodiment, the lubricious inner liner 210 may be of 0.0010″±0.0005″ thickness. In one embodiment, the lubricious inner liner 210 is thermally bonded to the reinforcement layer 240; the reinforcement layer 240 would ideally have a polymer thermoplastic inner surface (see FIG. 9B), or sufficient open wall (See FIG. 9B or FIG. 9C) allowing the soft wear resistant jacket polymer 220 to flow through the wall when melted during lamination at a temperature between 350 F to 450 F. The soft wear resistant jacket polymer 220 may be formed of any suitable polymeric material. In various embodiments, the soft wear resistant jacket polymer 220 may comprise a thermoplastic such as polyamide (e.g., nylon), polyurethane (PU), polyethylene terephthalate (PET), polyether block amide (PEBA), and may be comprised of multiple durometers with the softest being at the atraumatic tip and the harder being more proximal. In one embodiment, the soft wear resistant jacket polymer 220 may be of 0.002″±0.001″ thickness which may result in a final outside profile of 3.5 French (0.046″). In an additional embodiment, the soft wear resistant jacket polymer 220 may be even thicker to further improve durability/robustness, for example a soft wear resistant jacket polymer 220 thickness of 0.0045″ may result in a final outside profile of 3.89 French (0.051″). In one embodiment the reinforcement layer 240 may be a single piece stainless steel or nitinol tube formed into a coil from 1 or more strands of wire; in a second embodiment the reinforcement layer 240 may be a single piece metal tube of either nitinol or stainless steel which is modified with a laser cut pattern through the wall to improve the flexibility; in a third embodiment the reinforcement layer 240 may be comprised of a 2 over 1 pattern metal braided structure with several triaxial fibers bundles running longitudinal woven between the diamonds of the braided wire.

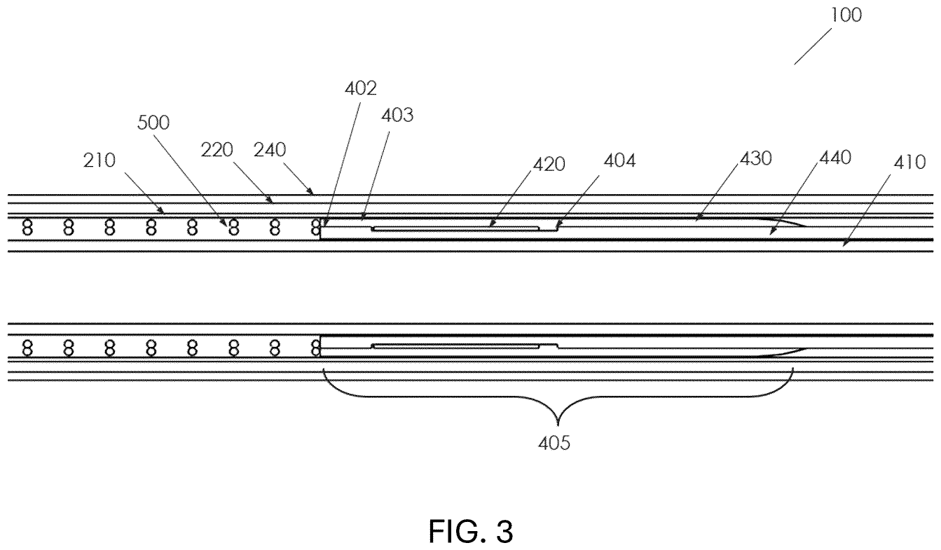

FIG. 3 illustrates a side cross sectional view along line B-B of the stent delivery system 100 shown in FIG. 1, showing the proximal portion of the self-expanding stent 500 present in the space allotted by the stabilizer 400 and the delivery sheath catheter 200. The delivery sheath catheter 200 is shown to be consistent with its three layers: the lubricious inner liner 210, the reinforcement layer 240, and the soft wear resistant jacket polymer 220. The stabilizer 400 herein comprises a flat face 402 in which the proximal end of stent 500 may bear load against, especially during deployment. Furthermore, the stabilizer 400 contains a collar length 403 that provides compressive load support during stent deployment, a radiopaque split marker band 420, that may be embedded into shaft recess 404 formed into reinforced shaft 440 and is encapsulated by a tip jacket 430. In one embodiment the radiopaque split marker band 420 is comprised of 90% platinum and 10% iridium. It is important to maintain low sliding forces, thus in one embodiment, the tip jacket 430 is smooth, cylindrical, and a low friction polymer. In one embodiment, the radiopaque split marker band 420 is cylindrical with a longitudinal cut through the wall of the part, allowing it to be assembled from the side of the shaft recess 404. The feature as a whole, made up flat face 402, collar length 403, radiopaque split marker band 420, shaft recess 404, and tip jacket 430, located over the tip jacket 430 is herein referred to as the stabilizer intermediate tip 405. In one embodiment, the reinforced shaft 440 may be braid reinforced with strands of 0.0005″×0.0025″ full hard 304 stainless steel flat wire arranged in a 90 pic per inch density woven to a 2 over 1 over 2 braid configuration, and embedded between an inner layer of stiff thermoset polymer such as polyimide and outer layer of stiff thermoset polymer such as polyimide; and the shaft recess 404 is created by laser ablation. The inner lumen 410 extends the full length of the stent delivery system as a single, continuous, uninterrupted piece.

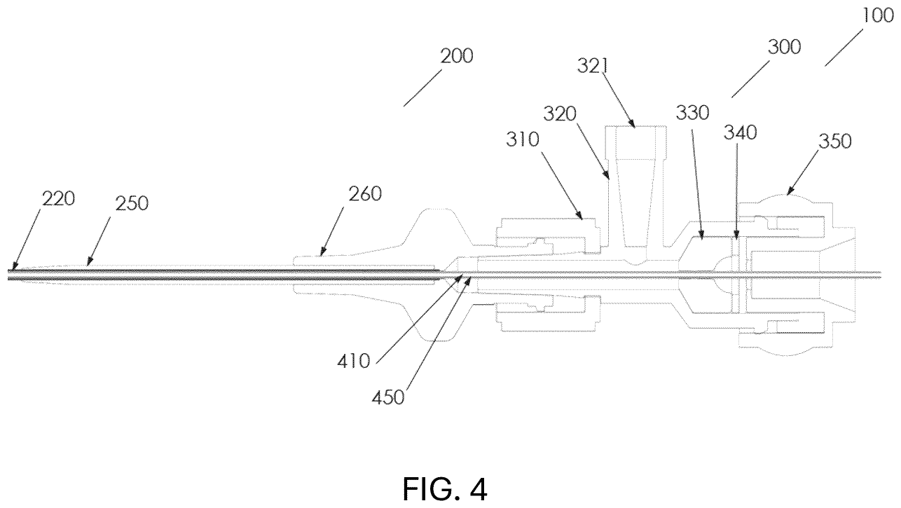

FIG. 4 illustrates a side cross sectional view along line C-C of the stent delivery system 100 shown in FIG. 1, containing the proximal side of the delivery sheath catheter 200 connected to the tuohy borst sideport 300, and mounted over the stabilizer 400. The soft wear resistant jacket polymer 220 terminates to a strain relief 250, which contains a molded hub 260. In one embodiment, the molded hub 260 with strain relief 250 are thermally bonded to the proximal end of the laminated shaft by connection to the soft wear resistant jacket polymer 220. The stabilizer 400 consists of an inner lumen 410 and a metallic cannular 450 that concentrically pass from the center of the delivery sheath catheter 200 and into the touhy borst sideport 300, where the metallic cannula 450 is sealed to a valve 330 before existing to open air. The tuohy borst sideport 300 comprises a tuohy body 320 that is mounted to a molded hub 260 by a Luer slip fit connection and held in place by a Luer thread between a nut 310 and the molded hub 260. A rotating nut 350 is threaded to the tuohy body 320, and may rotate to compress the valve 330, which clamps to an immobilize metallic cannular 450. The rotating nut 350 may be rotated in the opposite direction to allow movement of the metallic cannular 450. A lubricious washer 340 translates rotating loads to compressive loads. In one embodiment, the valve 330 is a low durometer silicone rubber, of shore A 30A durometer. In one embodiment, the tuohy body 320 contains a female Luer bore 321 molded to 80369-7:2016 or ISO 594-1 standard, and is used for flushing the interior of the tuohy body 320, and the space between the delivery sheath catheter 200 and the stabilizer 400, including the stent 500 with normal 0.9% saline before use using an off the shelf syringe. The inner lumen 410 extends the full length of the stent delivery system as a single, continuous, uninterrupted piece.

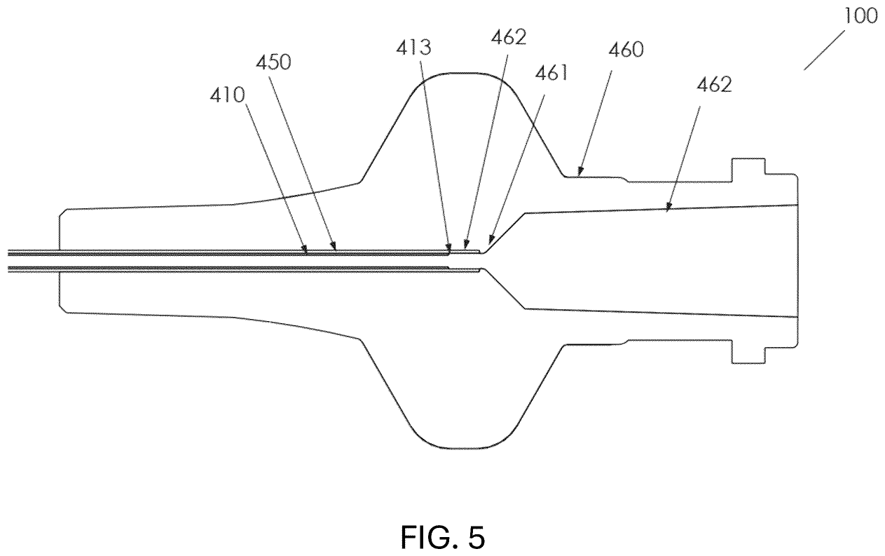

FIG. 5 illustrates a side cross sectional view along line D-D of the stent delivery system 100 shown in FIG. 1, showing the proximal portion of the stabilizer 400. The inner lumen 410 and metallic cannular 450 are connected to a stabilizer molded hub 460. In one embodiment, the cannula is grit blasted 462 to increase mechanical adhesion to the stabilizer molded hub 460 during an inert molding process and ensure a final tensile strength that exceeds 100N force. In one embodiment, in order to ensure low friction passage of a 0.014″ guidewire 700 (not shown), the inner lumen 410 is prepared with a proximal clean square cut end 413; and the stabilizer molded hub 460 has flash free smooth transition 461. In one embodiment, the stabilizer molded hub 460 contains a female Luer bore 462 molded to 80369-7:2016 OR ISO 594-1 standard that is used for flushing the guidewire lumen of the stent delivery with normal 0.9% saline before use.

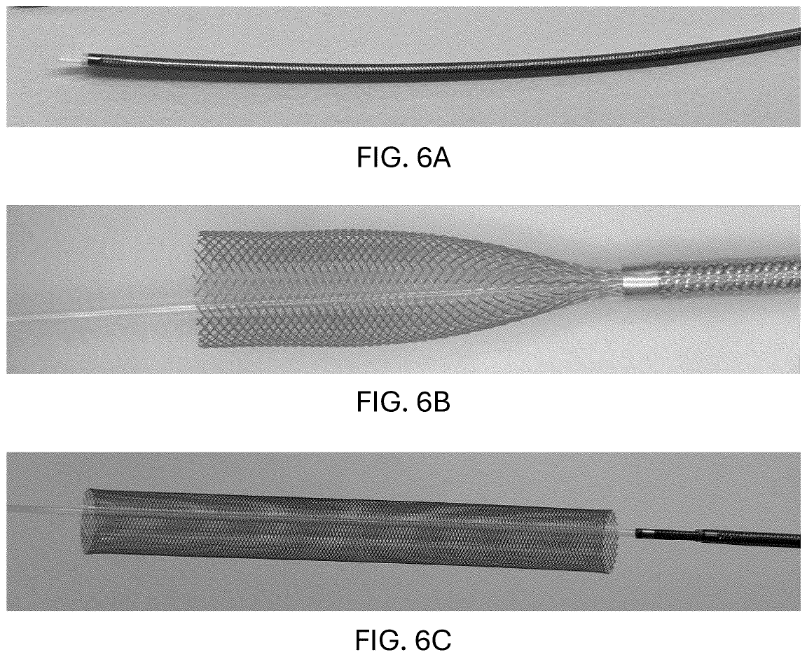

FIG. 6A is a perspective side view of the stent delivery system 100 shown in FIG. 1, with a stent 500 loaded in the distal end of the stent delivery system. The stabilizer 400 protrudes from the delivery sheath catheter 200, allowing it to be pushed onto a 0.014 in. guidewire.

FIG. 6B is a perspective side view of the stent delivery system 100 shown in FIG. 1, and state of operation in which the stent 500 in the loaded state FIG. 3A, has deployed partially from the distal end of the stent delivery system.

FIG. 6C is a perspective side view of the stent delivery system 100 shown in FIG. 1, with a stent 500 deployed from the end after having been loaded FIG. 3A and deployed partially FIG. 3B.

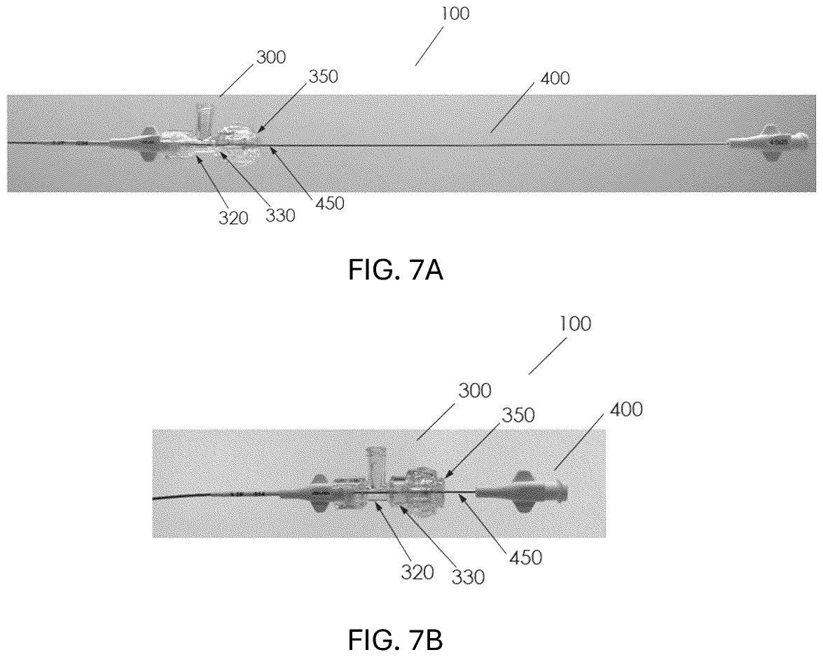

FIG. 7A is a perspective side view of the stent delivery system 100 shown in FIG. 1, proximal end with a stent 500 presumed to be loaded at the distal end. The touhy borst sideport 400 is shown in a state where the rotating nut 350 is threaded to the touhy body 320 in a tightened position, thus the valve 330 is compressed and holding the metallic cannular 450 in a clamped position.

FIG. 7B is a perspective side view of the stent delivery system 100 shown in FIG. 1, proximal end with a stent 500 presumed to be deployed at the distal end. The touhy borst sideport 400 herein contains the rotating nut 350 threaded to the touhy body 320, which may be loosened from a decompress valve 330 and freeing metallic cannular 450 to translate. A motion will transfer compressive load to the stent, deploying it from the stent delivery system.

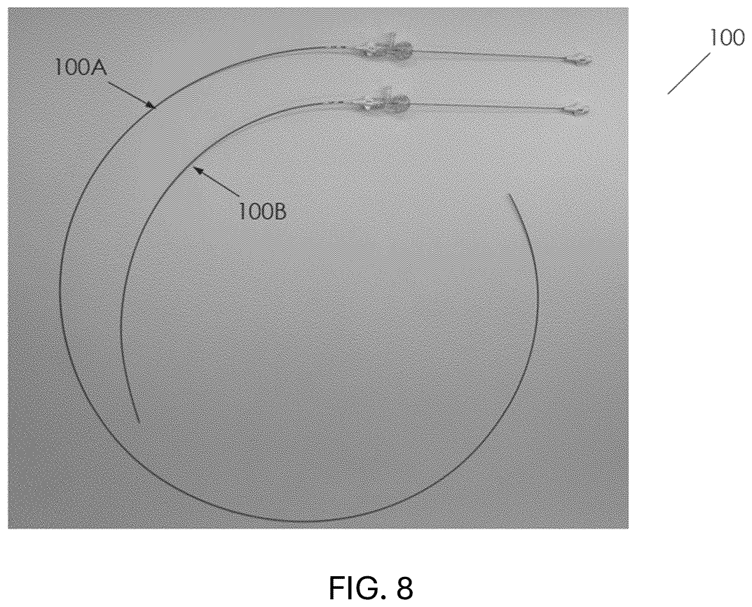

FIG. 8 illustrates stent delivery system 100 length embodiments. One stent delivery system 100 embodiment may be 120 CM length 100A. A 120 CM length may be intended to, but not limited to, accessing the bloodstream from a femoral access site and to navigate retrograde, over the lower arch, then antegrade through artery antegrade to below the knee sites.

One stent delivery system 100 embodiment may be 40 CM length 100B. A 40 CM length may be intended to, but not limited to, accessing the bloodstream from the foot and to navigate through artery retrograde to below the knee sites.

One stent delivery system 100 embodiment may be 220 CM length to enable femoral contralateral and reach distal lesions in the food below the knee.

One Stent Delivery System 100 embodiment may be 150 CM length to enable femoral retrograde and reach lesions in the brain.

FIG. 9A illustrates a delivery system sheath 200 embodiment where the reinforcement layer 240 is made of a stainless steel or nitinol tube formed into a coil from 1 or more strands of wire (240A). The reinforcement layer 240A is low thickness (between 0.0015-0.0020 thick), while still being high robustness.

FIG. 9B illustrates a delivery system sheath 200 embodiment where the reinforcement layer 240 is constructed of laser but hypotube (240B) ; further described as single piece metal tube of either super elastic nitinol or stainless steel which is modified with a laser cut spiral interrupt pattern through the wall to relieve stiffness and improve the flexibility. The reinforcement layer 240B is low thickness (between 0.0015-0.0020 thick), while still being high robustness.

FIG. 9C illustrates a delivery system sheath 200 embodiment where the reinforcement layer 240 is made of a braided skeleton tube (240C), wherein metal wires are oriented helically in a braided 2 over 1 pattern structure with triaxial polymer fiber bundles oriented axially, running longitudinal between the diamonds of the braided wire; a pair of polymer layers comprises a first polymer layer which encapsulates the inside surface of the braided skeleton tube, and wherein a second polymer layer encapsulates the outside surface of the braided skeleton tube. The reinforcement layer 240C is low thickness (between 0.0015-0.0020 thick), while still being high robustness.

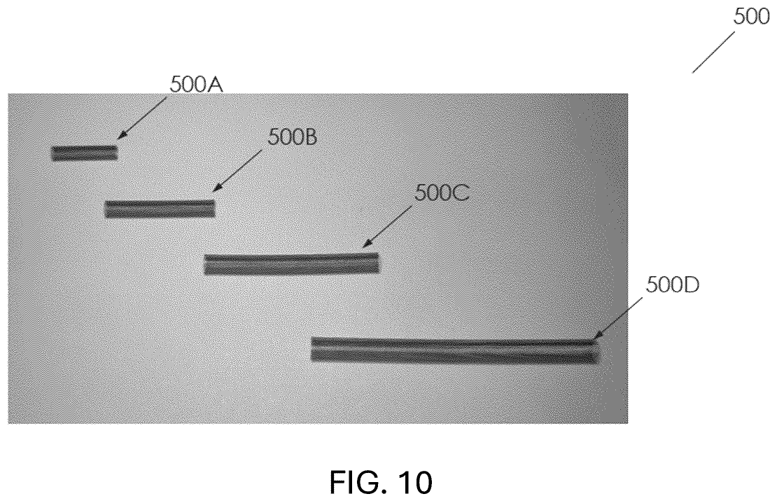

FIG. 10 illustrates various stent 500 diameter and length embodiments arranged on a flat surface: 3.0 mm diameter×15 mm long stent 500A, 3.5 mm diameter×25 mm long stent 500B, 4.0 mm diameter×40 mm long stent 500C, 4.5 mm diameter×60 mm long stent 500D. The stent diameter and length have varying loaded state lengths, which in various embodiments differ by the final lengths of the reinforced shaft 440.

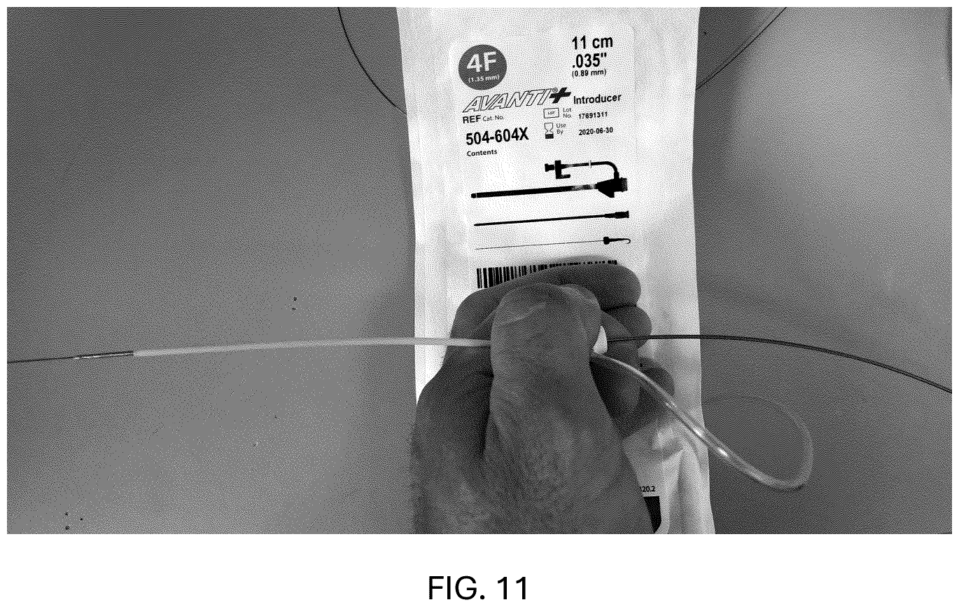

FIG. 11 is a perspective view of a stent delivery system, where the small crossing profile is sufficiently smaller than the internal diameter of an off-the-shelf 4 Fr Introducer Sheath allowing free passage.

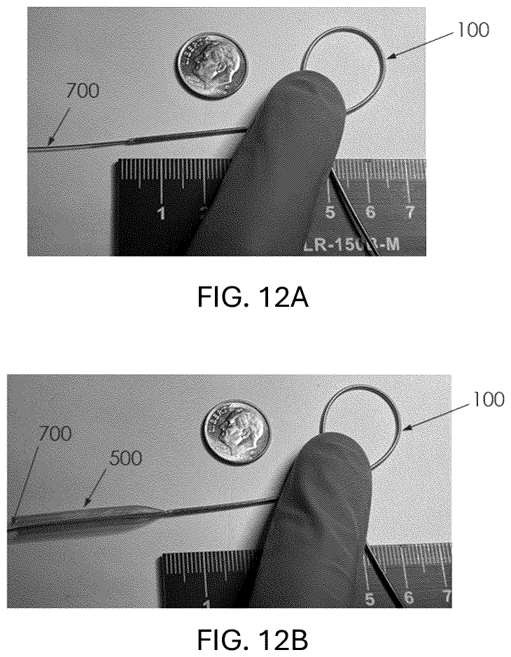

FIG. 12A is a perspective view of a stent delivery system 100 over a 0.014 guidewire 700, showing robustness and durability of the system being flexed aggressively to a diameter of 0.75 inches (19 mm), similar to the outside diameter of a U.S. dime, without sustaining damage such as kinking.

FIG. 12B is a perspective view of a stent delivery system 100 over a 0.014 guidewire 700, showing robustness and durability of the system being flexed aggressively to a diameter of 0.75 inches (19 mm), similar to the outside diameter of a U.S. dime, while reliably and dependably deploying a preloaded stent 500.

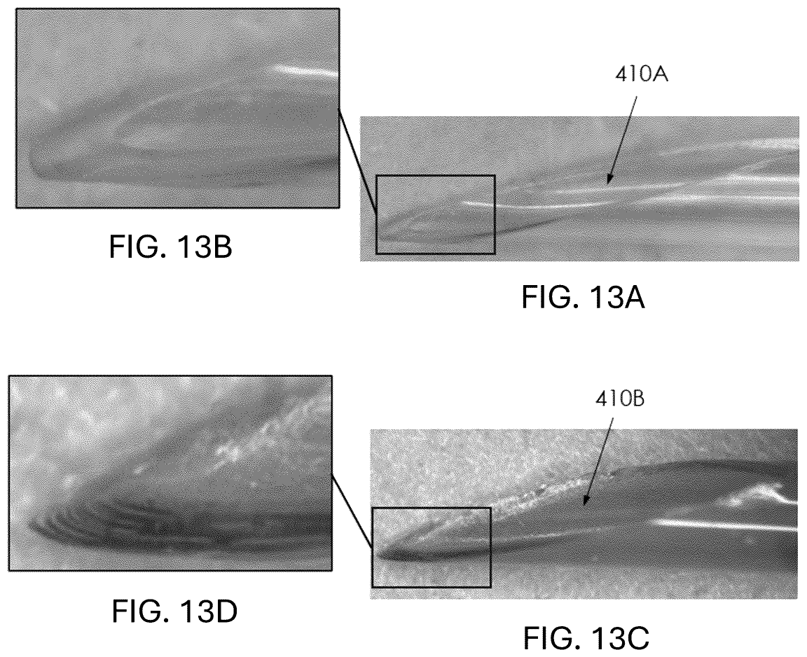

FIGS. 13A-13B show a perspective view of an inner lumen 410 of a preferred embodiment 410A, cut at 60° angle to show a singular solid-cross-section of a homogeneous thermoplastic polymer.

FIGS. 13C-13D show a perspective view of an inner lumen 410 of an alternative embodiment 410B, cut at 60° angle to show a layered cross-section of a dispensed thermoset polymer (e.g. polyimide). While the system is amenable to various modifications and alternative forms, specifics thereof have been shown by way of example in the drawings and will be described in detail. It should be understood, however, that the intention is not to limit the device to the particular embodiments described. On the contrary, the intention is to cover all modifications, equivalents, and alternatives falling within the spirit and scope of the system.

What is claimed is:

1. A stent delivery system comprised of:
(a) a robust delivery sheath catheter comprising: a lubricious inner liner formed of a lubricious polymeric material; a reinforcement layer configured to enhance flexibility and structural integrity; and an outer jacket formed of a wear-resistant polymeric material, the outer jacket including an atraumatic tip at the distal end;
(b) a stabilizer comprising: robust inner lumen configured to receive a guidewire having an inner diameter of approximately 0.0175 inches to fit freely with an 0.014 guidewire, the inner lumen extending from the proximal end to the distal end of the delivery sheath; and intermediate tip containing a flat face configured to bear a load of a preloaded stent during deployment; and a radiopaque split marker band embedded in a shaft recess of a stabilizer, and a radiopaque marker band being cylindrical with a longitudinal cut for side assembly;
(c) a self-expanding stent preloaded in a compressed state within a space defined between the lubricious inner liner of the delivery sheath catheter and the outer surface of inner lumen of the stabilizer, the stent positioned adjacent to a distal radiopaque marker band on the delivery sheath; and
(d) a tuohy borst sideport coupled to the proximal end of the delivery sheath, the sideport comprising: a body mounted to a molded hub of the delivery sheath catheter via a Luer slip fit connection; and a valve configured to selectively immobilize a metallic cannula of the stabilizer, the metallic cannula extending through the inner lumen and sealed to the valve;
wherein the system has a crossing profile between 3.20 Fr and 3.89 Fr and a length between 40 cm and 220 cm, and is configured to maintain structural integrity and responsiveness during introductory forces, to maintain structural integrity and responsiveness while advancing percutaneously through a vascular anatomy, to maintain structural integrity and responsiveness during deployment of a preloaded stent at a target, and to maintain structural integrity and responsiveness during stent delivery system withdrawal from a patient.

2. The stent delivery system of claim 1 consisting of an inner lumen material that is comprised of a singular solid cross-section of a homogeneous thermoplastic polymer, not a layered cross-section dispensed thermoset polymer (e.g. polyimide).

3. The stent delivery system of claim 1 comprising an inner lumen geometry that:
(a) has a generally circular internal open shape, with an internal diameter of 0.0175 inches±0.0005 inches, and a concentricity of at least 90% relative to its outer surface; and
(b) has an internal diameter compatible with a standard 0.014″ guidewire or an internal diameter configured to receive a 0.014″guidewire with minimal resistance and free movement.

4. The stent delivery system of claim 1 comprising an inner lumen construction that:
(a) extends the full length of the stent delivery system as a single, continuous, uninterrupted piece;
(b) has clean square-cut ends; and
(c) has an outer surface along at least a portion of its length configured to support a stent in a compressed state.

5. The stent delivery system of claim 1 comprising a stabilizer intermediate tip containing:
(a) a flat face to bear compressive stress of a loaded state stent during deployment;
(b) a collar length that provides support to the flat face during stent deployment;
(c) a radiopaque split marker band cut through the wall of the part, allowing it to be assembled from the side of the shaft recess;
(d) encapsulation provided by a smooth, cylindrical, and a low friction thermoplastic polymer jacket; and
(e) a reinforced shaft, having specific length configurations relevant to both the length of the stent in the loaded state, and to the stent overall delivery system length.

6. The stent delivery system of claim 1 comprising a delivery sheath catheter comprised of:
(a) a low-thickness, high-robustness reinforcement layer;
(b) a lubricious inner liner that is thermally bonded to the reinforcement layer;
(c) a wear-resistant polymer jacket thermally bonded to the reinforcement layer;
(d) a laminated shaft structure in which the reinforcement layer, lubricious liner, wear-resistant polymer jacket, and a radiopaque marker band are thermally laminated together using a fluorinated ethylene propylene (FEP) shrink tube and heat exposure, the laminated shaft being configurable according to stent delivery system length specifications;
(e) hub with strain relief thermally bonded to the proximal end of the laminated shaft; and
(f) a radiopaque marker band positioned at the distal end of the delivery sheath catheter, the marker band having an outer diameter coincident with the outer diameter of the reinforcement layer.
